(19) United States
(12) Patent Application Publication
(10) Pub. No.: US 2017/0119257 A1
(43) Pub. Date: May 4, 2017

(54) MEASUREMENT APPARATUS AND MEASUREMENT METHOD

(71) Applicant: KYOCERA Corporation, Kyoto (JP)

(72) Inventor: Kinya SUGIMOTO, Okaya-shi, Nagano (JP)

(73) Assignee: KYOCERA Corporation, Kyoto (JP)

(21) Appl. No.: 15/320,161

(22) PCT Filed: Jun. 22, 2015
(86) PCT No.: PCT/JP2015/003117

§ 371 Date: Dec. 19, 2016

(30) Foreign Application Priority Data
Jun. 24, 2014 (JP) 2014-129215

(51) Int. Cl. A61B 5/026; A61B 5/00

(52) U.S. Cl. CPC A61B 5/0261; A61B 5/6826; A61B 5/6898

(57) ABSTRACT

A measurement apparatus for measuring biological information when a test site contacts with a contact interface includes a biological sensor for acquiring a biometric output from the test site, a color information acquisition unit for acquiring color information of the test site in contact with the contact interface, a notification interface, and a controller, wherein the controller, based on the color information, causes the notification interface to notify of information about a contact condition of the test site in contact with the contact interface and, based on the biometric output, measures the biological information.

### CROSS REFERENCE TO RELATED APPLICATION

This application claims priority to and the benefit of Japanese Patent Application No. 2014-129215 (filed on Jun. 24, 2014), the entire contents of which are incorporated herein by reference.

### TECHNICAL FIELD

This disclosure relates to a measurement apparatus and a measurement method.

### BACKGROUND

Conventionally, there is known an apparatus for measuring biological information by acquiring information output from a living body from a test site such as a subject's (user's) fingertip or the like.

### SUMMARY

A measurement apparatus of the disclosure herein is a measurement apparatus for measuring biological information when a test site contacts with a contact interface, the measurement apparatus including:

a biological sensor for acquiring a biometric output from the test site;

a color information acquisition unit for acquiring color information of the test site in contact with the contact interface;

a notification interface; and

a controller, wherein

the controller, based on the color information, causes the notification interface to notify of information about a contact condition of the test site in contact with the contact interface and, based on the biometric output, measures the biological information.

It is to be understood that a method substantially corresponding to the measurement apparatus described above may implement the disclosure herein and thus is included in the scope of the disclosure herein.

For example, a measurement method of the disclosure herein includes:

in measuring biological information when a test site contacts with a contact interface,

acquiring with a color information acquisition unit, color information of the test site in contact with the contact interface;

acquiring with a biological sensor, a biometric output from the test site; and

causing with a controller, based on the color information, the notification interface to notify of information about a contact condition of the test site in contact with the contact interface and, measuring, based on the biometric output, the biological information.

### BRIEF DESCRIPTION OF THE DRAWINGS

In the accompanying drawings:

FIG. 1 is a functional block diagram illustrating a schematic configuration of a measurement apparatus according to one embodiment of the disclosure herein;

FIG. 2 is a diagram illustrating an example of a using condition of the measurement apparatus of FIG. 1;

FIG. 3 is a flowchart illustrating an example of a measurement operation of blood flow performed by a controller of FIG. 1; and

FIGS. 4A and 4B are diagrams illustrating an example of a mobile phone having the measurement apparatus of FIG. 1 mounted thereon.

### DETAILED DESCRIPTION

For example, a known blood flow measurement apparatus that measures blood flow as the biological information irradiates a fingertip with laser light and measures the blood flow based on scattered light from the blood flow in a capillary at the fingertip.

Accuracy in the measurement of the biological information depends on a condition of the capillaries in the test site, and the condition of the capillaries changes based on a contact condition of the test site in contact with the measurement apparatus. Therefore, the measurement apparatus, for highly accurate measurement of the biological information, is desired to make the user contact with the test site in a suitable contact manner.

Therefore, it could be helpful to provide a measurement apparatus and a measurement method which are capable of improving the accuracy in the measurement of the biological information.

Hereinafter, an embodiment of the disclosure herein will be described in detail with reference to the drawings.

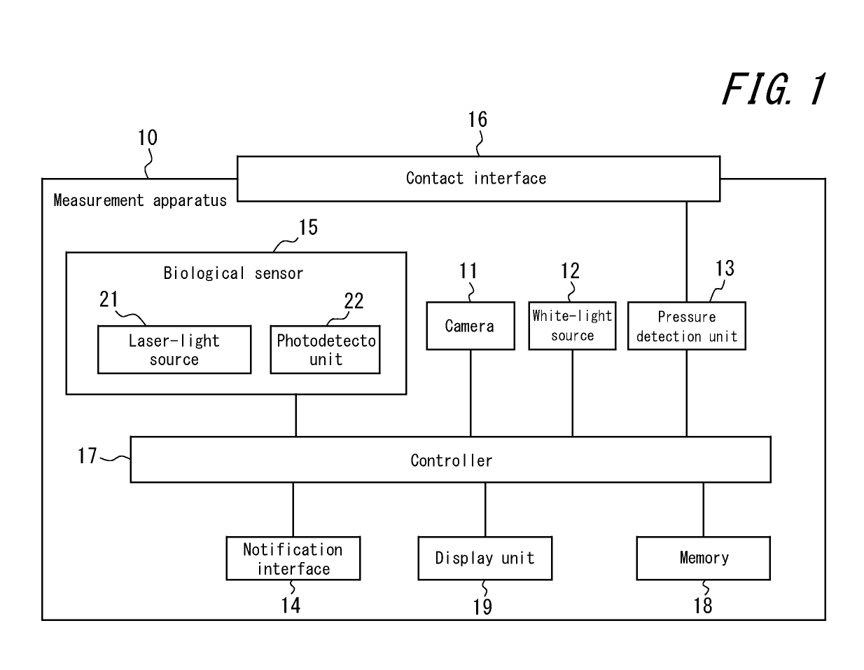

FIG. 1 is a functional block diagram illustrating a schematic configuration of a measurement apparatus according to one embodiment of the disclosure herein. A measurement apparatus 10 includes a camera 11, a white-light source 12, a pressure detection unit 13, a notification interface 14, a biological sensor 15, a contact interface 16, a controller 17, a memory 18, and a display unit 19.

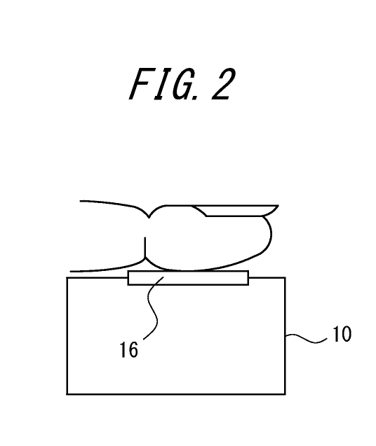

The measurement apparatus 10 acquires a biometric output of a subject in contact with the contact interface 16 by using the biological sensor 15 and measures biological information based on the biometric output. FIG. 2 is a diagram illustrating an example of a using condition of the measurement apparatus 10 in which the subject is pressing the finger serving as a test site against the contact interface 16 of the measurement apparatus 10. The measurement apparatus 10, when the finger is pressed against the contact interface 16 as illustrated in FIG. 2, acquires the biometric output by using the biological sensor 15 and measures the biological information.

The biological information measured by the measurement apparatus 10 may be any biological information measurable by the biological sensor 15. According to the present embodiment, the measurement apparatus 10 measures, by way of example, blood flow of the subject serving as information about blood flow, and the following is a description thereof.

In FIG. 1, the camera 11 acquires color information of the test site in contact with the contact interface 16. The camera 11 serves as an example of a color information acquisition unit and is, for example, a digital video camera in the present embodiment. The color information acquisition unit is not limited to the camera 11 but may have any configuration capable of acquiring the color information such as, for example, a color sensor using LED (Light Emitting Diode) and the like. The color information acquisition unit may particularly acquire the color information of red components of the test site.

The white-light source 12 emits white light to the test site. The camera 11 acquires the color information of the white light reflected on the test site after emitted from the white-light source 12. When the camera 11 has a function to be able to acquire the color information without the white light emitted to the test site, the measurement apparatus 10 does not need to include the white-light source 12.

The pressure detection unit 13 detects a contact pressure applied to the contact interface 16 by the test site. The pressure detection unit 13 may be constituted by using, for example, a piezoelectric element. The pressure detection unit 13 is connected to the controller 17 and transmits, to the controller 17, a pressure signal indicative of the contact pressure detected. Accordingly, the pressure detection unit 13, when the test site is in contact with the contact interface 16, detects the contact pressure applied to the contact interface 16 by the test site and transmits the pressure signal indicative of the contact pressure detected to the controller 17. The controller 17 may determine whether the test site is in contact with the contact interface 16 based on the pressure signal.

The notification interface 14, under control by the controller 17, notifies of information about a contact condition of the test site to the contact interface 16. The information about the contact condition is, for example, information about whether the test site is in contact with the contact interface 16 in such a manner that the capillaries in the test site are in a condition suitable for the measurement of the biological information.

The notification interface 14 may perform the notification by employing, for example, a visual manner using an image, a character, lighting, and the like, or an auditory manner using a voice and the like, or a combination thereof. The notification interface 14, in order to notify by employing the visual manner, displays, for example, the image or the character in the display unit 19. The notification interface 14 may notify by, for example, turning on a light emitter such as the LED and the like. The notification interface 14, in order to notify by employing the auditory manner, outputs, for example, an alarm sound, a voice guidance, and the like from a sound generating device such as a speaker. The notification issued by the notification interface 14 is not limited to the visual manner and the auditory manner but may by any manner perceivable by the subject. The control of the notification interface 14 by the controller 17 will be described in detail later.

The biological sensor 15 acquires the biometric output from the test site. When the measurement apparatus 10 measures the blood flow as described in the present embodiment, the biological sensor 15 includes a laser-light source 21 and a photodetector unit 22.

The laser-light source 21 emits a laser light under control by the controller 17. The laser-light source 21 emits a measurement light which is, for example, a laser light in a wavelength which allows detection of a predetermined component contained in the blood and may be, for example, an LD (Laser Diode).

The photodetector unit 22 receives, as the biometric output, scattered light of the measurement light from the test site. The photodetector unit 22 is constituted by using, for example, a PD (Photodiode). The biological sensor 15 transmits a photoelectric conversion signal of the scattered light received by the photodetector unit 22 to the controller 17.

The contact interface 16 is a unit to be touched by the test site such as the finger or the like of the subject for measurement of the biological information. The contact interface 16 may be, for example, a plate-like member. The contact interface 16 is made of a transparent member to receive at least the measurement light from the laser-light source 21, the white light from the white-light source 12, and the scattered light of the measurement light and reflected light of the white light both from the test site in contact with the contact interface 16.

The controller 17 is a processor for controlling and managing the measurement apparatus 10 in its entirety including each functional block thereof. The controller 17 is constituted by using a processor such as CPU (Central Processing Unit) for executing a program defining control procedure stored in, for example, the memory 18 or an external storage medium.

The controller 17 causes the notification interface 14 to notify of the information about the contact condition of the test site in contact with the contact interface 16. The controller 17 controls a notification such that the subject may adjust the contact condition in order to have the capillaries in the test site in the condition suitable for the measurement of the biological information. For example, when the test site applies too much contact pressure to the contact interface 16, the capillaries in the test site become squashed and inhibit, or reduce, the blood flow therein. The measurement of the blood flow in such a condition inhibits an output of a highly accurate result of the measurement of the blood flow. In this case, the controller 17 causes the notification interface 14 to notify the subject of that it is necessary to reduce the contact pressure applied by the test site.

The controller 17 determines the contact condition based on the color information acquired from the camera 11. A human finger with no pressure applied thereto allows blood flow in the capillaries and thus contains the red components more than those when the finger with a pressure applied thereto. When a pressure is applied to the finger, the blood flow in the capillaries decreases, reducing a ratio of the red components in the test site. The controller 17 utilizes such a feature of the color of the finger and determines the contact condition based on the ratio of the red components in the color information. Note that the red components are in wavelengths of 610 nm to 780 nm.

Greater the pressure applied to the finger, the less blood flows in the capillaries, reducing the ratio of the red components in the test site. The controller 17 of the measurement apparatus 10 according to the present embodiment utilizes this feature and determines the contact condition based particularly on a change in the ratio of the red components. The controller 17, when determining the contact condition based on the change in the ratio of the red components in this manner, determines the change in the ratio of the red components in the test site by using, as a reference, the ratio of the red components in the test site when the subject contacts with the contact interface 16 after setting the measurement apparatus 10 to a condition capable of measuring the biological information.

The controller 17 determines whether the test site is in contact with the contact interface 16, based on whether the pressure detection unit 13 detects a pressure. The controller 17 acquires an image (an initial image) captured by the camera 11 at the time of detection of the pressure by the pressure detection unit 13. The controller 17, by analyzing the color information of the initial image thus acquired, calculates the ratio of the red components contained in the image. Subsequently, the controller 17, while the biological sensor 15 is acquiring the biometric output, continuously acquires images captured by the camera 11 and analyzes the color information of the images, thereby calculating the ratios of the red components contained in the images. The controller 17, when the ratio of the red components calculated is smaller than the ratio of the red components in the initial image by a predetermined ratio or more, controls the notification interface 14 to issue an instruction to reduce the contact pressure. The predetermined ratio used as a determination criterion whether to issue the instruction is preliminarily stored in, for example, the memory 18.

The controller 17, at the start of the measurement of the biological information, may determine whether the blood flow can be measured, based on the image captured by the camera 11. The controller 17, when, for example, the ratio of the red components calculated based on the initial image is equal to or lower than a predetermined threshold, determines that an amount of the blood flowing in the test site is insufficient for the measurement of the blood flow. In this case, the controller 17 displays, in the display unit 19, an indication that the blood flow cannot be measured. The controller 17 may display, in the display unit 19, a list of possible causes of the amount of flowing blood insufficient for the measurement of the blood flow. The possible causes include, for example, Raynaud's disease of the subject, the test site not at a height similar to the heart, and the like. The Raynaud's disease is a disease which stops flow of the arterial blood due to spasmodic contraction of the blood vessels. When the subject has the Raynaud's disease, the blood does not flow in the finger, and thus the controller 17 determines that the blood flow cannot be measured. Also, when the test site is not at the height similar to the heart such as when the test site is in contact with the contact interface 16 while the subject is holding the hand up, a hydrostatic pressure in the test site is lowered, reducing the blood flow in the test site. In this case, the controller 17 determines that the amount of the flowing blood is insufficient for the measurement of the blood flow.

The controller 17, when a problem due to the possible cause is solvable, may display a solution to the problem in addition to the possible cause in the display unit 19. For example, when the blood flow cannot be measured due to the test site which is not at the height similar to the heart, the display unit 19 may display a message such as, for example, “Please move the finger to the height similar to the heart for the measurement”.

The controller 17, when displaying the indication that the blood flow cannot be measured, displays a selection panel for allowing selection whether to retry the measurement of the biological information in the display unit 19. When the subject, by using an input unit of the measurement apparatus 10, selects to retry the measurement in the selection panel, the controller 17 once again determines whether the blood flow can be measured based on the image captured by the camera 11. When the subject, by using the input unit, selects not to retry the measurement in the selection panel, the controller 17 ends without measuring the biological information. The controller 17, by allowing the subject to select whether to retry the measurement in this manner, may end the measurement when, for example, the subject is aware of having the Raynaud's disease, or retry the measurement after the subject has overcome the possible cause.

The controller 17, when determining that the blood flow can be measured based on the image captured by the camera 11, causes the laser-light source 21 to emit the laser light. After the biological sensor 15 starts acquiring the biometric output from the laser light emitted, the controller 17 determines whether the biological sensor 15 has finished acquiring the biometric output. For example, the controller 17 may determine that the biological sensor 15 has finished acquiring the biometric output when a predetermined period of time has passed from the start of the acquisition of the biometric output by the biological sensor 15. Alternatively, for example, the controller 17 may determine that the acquisition of the biometric output is finished when the biometric output in a sufficient quantity for the measurement of the biological information is acquired by the biological sensor 15. The controller 17, when determining that the acquisition of the biometric output is finished, causes the laser-light source 21 to stop emitting the laser light. In this manner, the controller 17 controls the acquisition of the biometric output by the biological sensor 15.

The controller 17 measures the biological information based on the biometric output acquired by the biological sensor 15. In particular, the controller 17 generates the biological information based on an output (a biological information output) from the photodetector unit 22.

Here, a blood flow measuring technology using a Doppler shift employed by the controller 17 will be described. The controller 17, in order to measure the blood flow, causes the laser-light source 21 to emit the laser light into tissues of a living body (the test site) such that the photodetector unit 22 receives the scattered light from the inside of the tissue of the living body. Then, the controller 17 calculates the blood flow based on an output associated with the scattered light received.

Inside the tissues of the living body, the scattered light scattered by the blood cells which are moving is subjected to a frequency shift (the Doppler shift) due to Doppler effect in proportion to a moving speed of the blood cells in the blood. The controller 17 detects an Unari-signal (also referred to as a beat signal) generated by optical interference between the scattered light from still tissues and the scattered light from the blood cells which are moving. The beat signal indicates intensity represented by a time function. Then, the controller 17, from the beat signal, generates a power spectrum in which power is represented by a frequency function. In the power spectrum of the beat signal, the Doppler shift frequency is in proportion to the moving speed of the blood cells, and the power corresponds to an amount of the blood cells. Then, the controller 17 calculates the blood flow by multiplying the power spectrum of the beat signal by the frequency and integrating.

The controller 17 displays the biological information measured in the display unit 19.

The memory 18 may be constituted by using a semiconductor memory, a magnetic memory, and the like and stores various information and a program for operating the measurement apparatus 10, as well as functioning as a work memory. The memory 18 stores, for example, the threshold, the ratio and the like serving as the criteria used by the controller 17 for the determination. The memory 18 may store a history of the blood flow measured by the measurement apparatus 10. The memory 18 may store the ratio of the red components in the test site in contact with the contact interface 16 which is used as the criterion by the controller 17 when determining a change ratio of the red components in the test site.

The display unit 19 is a display device constituted by using a known display such as a liquid crystal display, an organic EL display, an inorganic EL display, or the like. The display unit 19 may display various information under the control of the controller 17 and displays, for example, the biological information measured.

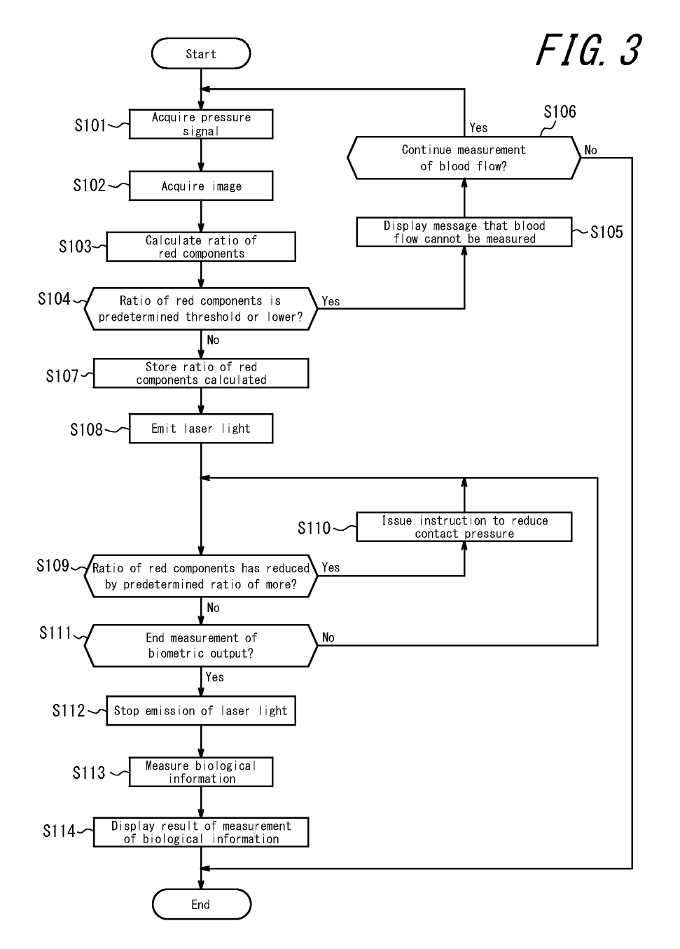

Next, an example of a measurement operation of the blood flow performed by the controller 17 will be described with reference to a flowchart illustrated in FIG. 3. The controller 17 starts the flow of FIG. 3 when the subject operates the measurement apparatus 10 and thus the measurement apparatus 10 enters into a state capable of measuring the biological information.

The controller 17 acquires the pressure signal output by the pressure detection unit 13 when the subject brings the test site into contact with the contact interface 16 (step S101). Thereby, the controller 17 recognizes that the test site is in contact with the contact interface 16.

The controller 17 acquires the image captured by the camera 11 when the test site contacts with the contact interface 16 (step S102).

The controller 17 analyzes the color information of the image acquired at step S102 and calculates the ratio of the red components in the image (step S103).

The controller 17 determines whether the ratio of the red components calculated is equal to or lower than the predetermined threshold stored in the memory 18 (step S104).

The controller 17, when determining that the ratio of the red components is equal to or lower than the predetermined threshold (Yes at step S104), determines that the amount of the flowing blood is insufficient for the measurement of the blood flow and displays the indication that the blood flow cannot be measured in the display unit 19 (step S105). At this time, the controller 17 may display, in the display unit 19, the possible cause of the amount of the flowing blood insufficient for the measurement of the blood flow and the solution to the problem due to the cause.

The controller 17 displays, in the display unit 19, the selection panel asking whether to retry the measurement of the blood flow, i.e., whether to continue the measurement of the blood flow in a present procedure. The controller 17, based on the input to the display by the subject, determines whether to continue the measurement of the blood flow (step S106).

The controller 17, when determining not to continue the measurement of the blood flow based on the input by the subject (No at step S106), ends the procedure without measuring the blood flow.

The controller 17, when determining to continue the measurement of the blood flow based on the input by the subject (Yes at step S106), proceeds to step S101 and acquires the pressure signal output by the pressure detection unit 13 when the test site contacts with the contact interface 16.

The controller 17, when determining at step S104 that the ratio of the red components is higher than the predetermined threshold (No at step S104), stores the ratio of the red components calculated at step S103 in the memory 18 (step S107).

The controller 17 causes the laser-light source 21 to emit the laser light (step S108). Thereby, the controller 17 causes the biological sensor 15 to start acquiring the biometric output.

While the biological sensor 15 is acquiring the biometric output, the controller 17 analyzes the color information of the image captured by the camera 11 and determines whether the ratio of the red components has reduced by the predetermined ratio stored in the memory 18 or more (step S109).

The controller 17, when determining that the ratio of the red components has reduced by the predetermined ratio or more (Yes at step S109), determines that the capillaries are squashed due to the high contact pressure applied to the contact interface 16 by the test site and causes the notification interface 14 to issue the instruction to reduce the contact pressure (step S110). The subject, when recognizing the instruction, reduces the pressure of the test site. Then, the controller 17 proceeds to step S109 and determines whether the ratio of the red components in the test site has reduced by the predetermined ratio or more.

The controller 17, when determining that the ratio of the red components in the test site has not reduced by the predetermined ratio or more (No at step S109), determines whether the biological sensor 15 has finished acquiring the biometric output (step S111).

The controller 17, when determining that the biological sensor 15 has not finished acquiring the biometric output (No at step S111), proceeds to step S109.

The controller 17, when determining that the biological sensor 15 has finished acquiring the biometric output (Yes at step S111), causes the laser-light source 21 to stop emitting the laser light (step S112).

The controller 17 measures the biological information based on the biometric output acquired by the biological sensor 15 (step S113).

The controller 17 displays, in the display unit 19, the result of the measurement of the biological information measured at step S113 (step S114). The subject may know the blood flow by viewing the result of the measurement thus displayed.

According to the measurement apparatus 10 of the present embodiment, as described above, the color information acquisition unit acquires the color information of the test site in contact with the contact interface 16, and the controller 17, based on the color information, determines whether the capillaries in the test site are in the suitable condition for the measurement of the biological information, i.e., whether the capillaries are not squashed. The controller 17, when determining that the capillaries are squashed, causes the notification interface 14 to notify the subject of the instruction to reduce the contact pressure. Therefore, the subject may easily adjust the contact pressure in such a manner that the capillaries become the suitable condition for the measurement of the biological information. Accordingly, since the measurement apparatus 10 may easily measure the biological information when the capillaries are in the suitable condition for the measurement of the biological information, the accuracy in the measurement of the biological information may be improved.

It is to be understood that the disclosure herein is not limited to the above embodiment but may be modified or changed in various manners. For example, a function and the like included in each constituent, each step, and the like may be rearranged avoiding logical inconsistency, so as to combine a plurality of constituents, steps, and the like together or to separate them.

For example, although in the above embodiment the controller 17, when the pressure detection unit 13 detects the pressure, determines that the test site is in contact with the contact interface 16, the determination about whether the test site is in contact with the contact interface 16 does not necessarily need to be based on the detection by the pressure detection unit 13. The controller 17 may determine that the test site is in contact with the contact interface 16 based on, for example, the image captured by the camera 11. Before the test site contacts with the contact interface 16, the camera 11 captures an image around the measurement apparatus 10 and, when the test site is in contact with the contact interface 16, captures an image of the test site. As described above, the image captured by the camera 11 greatly changes between before and after the test site contacts with the contact interface 16. The controller 17, by utilizing this feature, determines whether the test site is in contact with the contact interface 16 based on the image captured by the camera 11. In particular, the controller 17 performs an image analysis of the image captured by the camera 11. The controller 17, when the image is greatly changed, determines that the test site is in contact with the contact interface 16. In this case, the measurement apparatus 10 does not need to include the pressure detection unit 13.

Although in the above embodiment the controller 17 causes the notification interface 14 to issue the notification when the ratio of the red components is reduced by predetermined ratio or more with respect to the ratio of the red components in the test site in the initial image, the notification issued by the notification interface 14 is not limited to this manner. For example, the controller 17 may cause the notification interface 14 to notify when the ratio of the red components in the test site is lower than a predetermined lower limit threshold. The controller 17, when the ratio of the red components in the test site is lower than the predetermined lower limit threshold, determines that the capillaries are squashed and the amount of the flowing blood is insufficient for the measurement of the blood flow. The controller 17, based on such a determination, causes the notification interface 14 to issue the instruction to reduce the contact pressure.

Further, the controller 17 may cause the notification interface 14 to notify when the ratio of the red components in the test site is higher than a predetermined upper limit threshold. When the ratio of the red components is higher than the predetermined upper limit threshold, the contact pressure of the test site applied to the contact interface 16 is small, possibly causing the biometric output acquired by the biological sensor 15 to include noise and deteriorating the accuracy in the measurement of the biological information measured by the controller 17. To prevent such a deterioration of the accuracy in the measurement of the biological information, the controller 17 may control the notification based on the upper limit threshold. In particular, when the ratio of the red components in the test site is higher than the predetermined upper limit threshold, the controller 17 causes the notification interface 14 to issue an instruction to increase the contact pressure.

The controller 17, both when the ratio of the red components in the test site is lower than the predetermined lower limit threshold and when the ratio is higher than the upper limit threshold, may cause the notification interface 14 to issue an instruction to adjust the contact pressure appropriately.

The predetermined lower limit threshold and the predetermined upper limit threshold may be preliminarily stored in, for example, the memory 18. Alternatively, the predetermined lower limit threshold and the predetermined upper limit threshold may be determined by the controller 17 by performing calibration for each subject. In performing the calibration, the predetermined lower limit threshold and the predetermined upper limit threshold reflect individual differences in, for example, a thickness of the skin in the test site, a size of the capillaries, and the like. Therefore, the measurement apparatus 10 may measure the biological information more accurately.

The measurement apparatus 10 may include a temperature sensor for measuring ambient temperature (temperature) of the measurement apparatus 10 and, based on the temperature measured by the temperature sensor, change at least one of the predetermined lower limit threshold and the predetermined upper limit threshold of the ratio of the red components in the test site. For example, when the ambient temperature of the measurement apparatus 10 is lower than a predetermined temperature threshold, the subject may have a poor blood circulation. Therefore, the measurement apparatus 10 may set at least one of the predetermined lower limit threshold and the predetermined upper limit threshold to be lower than those set when the temperature is equal to or higher than the predetermined temperature threshold.

The controller 17 may cause the laser-light source 21 to emit the laser light based on the contact condition determined on the basis of the ratio of the red components in the test site. For example, the controller 17 may control to emit the laser light when determining that the test site is in contact with the contact interface 16 in the suitable condition for the measurement of the biological information, and control to stop the emission of the laser light when determining that the contact condition is no longer in the suitable condition (for example, Yes at step S109 of the flowchart in FIG. 3). When the controller 17 controls the emission of the laser light based on the contact condition in this manner, unnecessary power consumption may be suppressed.

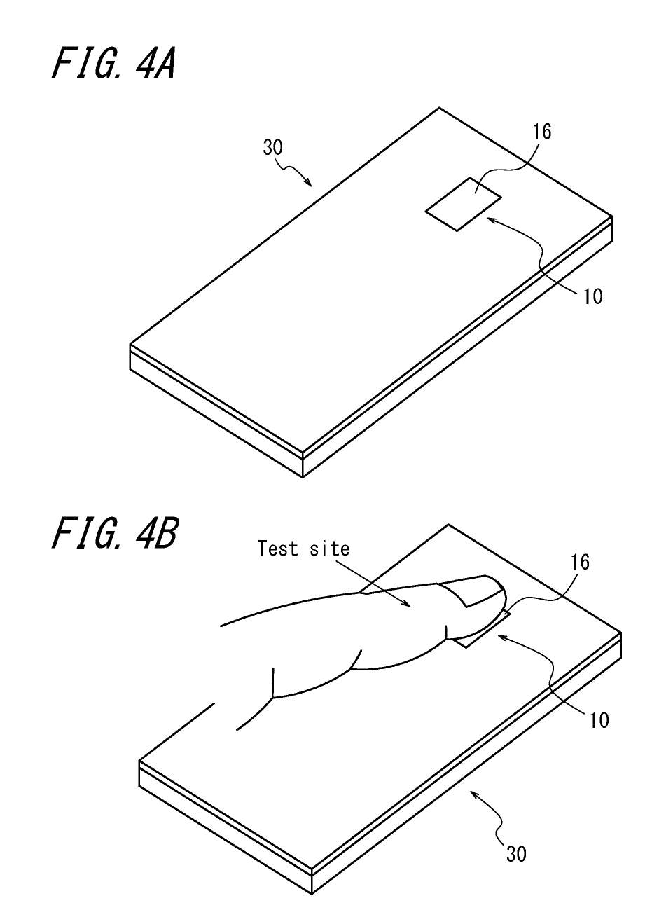

The measurement apparatus 10 according to the above embodiment may be mounted on various electronic apparatuses. FIGS. 4A and 4B are diagrams illustrating an example of a mobile phone having the measurement apparatus 10 of FIG. 1 mounted thereon. As illustrated in FIG. 4A, a mobile phone 30 has the measurement apparatus 10 on a rear side thereof.

FIG. 4B is a diagram illustrating an example when the user measures the biological information by using the mobile phone 30 having the measurement apparatus 10 mounted thereon. The subject brings the finger into contact with the contact interface 16 of the measurement apparatus 10 such that the measurement apparatus 10 measures the biological information.

When the subject measures the biological information by using the mobile phone 30 having the measurement apparatus 10 mounted thereon, the measurement apparatus 10 may start the measurement of the biological information in response to activation of a dedicated application for the measurement of the biological information by the subject using the mobile phone 30. The measurement apparatus 10 may automatically start the measurement of the biological information when detecting the contact pressure applied to the contact interface 16. In this case, the subject may start the measurement of the biological information simply by bringing the finger into contact with the contact interface 16, without activating the application.

When the measurement apparatus 10 is mounted on the electronic apparatus as illustrated in FIGS. 4A and 4B, each function unit of the electronic apparatus may have the function of each function unit of the measurement apparatus 10 illustrated in FIG. 1. For example, the measurement apparatus 10 may use a camera of the mobile phone 30 as the camera 11, or a display of the mobile phone 30 as the display unit 19.

Arrangement of the measurement apparatus 10 on the mobile phone 30 is not limited to that illustrated in FIGS. 4A and 4B. For example, the measurement apparatus 10 may be arranged somewhere else on the rear side of the mobile phone 30, or on a front side or a lateral side of the mobile phone 30.

The electronic apparatus having the measurement apparatus 10 mounted thereon is not limited to the mobile phone 30. The measurement apparatus 10 may be mounted on various electronic apparatuses including, for example, a portable music player, a laptop computer, a watch, a tablet computer, a gaming machine, and the like.

Further, although in the above embodiment the controller 17 of the measurement apparatus 10 generates the biological information based on the output of the photodetector unit 22, the generation of the biological information is not limited to this manner. For example, a server apparatus connected to the measurement apparatus 10 in a wired manner, in a wireless manner, or a combination thereof, may have a function unit corresponding to the controller 17, and generate the biological information. In this case, the measurement apparatus 10 acquires the biological information output from the biological sensor 15 and transmits the biological information output thus acquired to the server apparatus via a communication unit separately provided. Then, the server apparatus generates the biological information based on the biological information output and transmits the biological information thus generated to the measurement apparatus 10. The subject may view the biological information received by the measurement apparatus 10 by operating to display the biological information in the display unit 19. When the server apparatus generates the biological information in this manner, the measurement apparatus 10 may be downsized in comparison to that having all function units as illustrated in FIG. 1.

1. A measurement apparatus for measuring biological information when a test site contacts with a contact interface, the measurement apparatus comprising:
a biological sensor for acquiring a biometric output from the test site;
a color information acquisition unit for acquiring color information of the test site in contact with the contact interface;
a notification interface; and
a controller, wherein
the controller, based on the color information, causes the notification interface to notify of information about a contact condition of the test site in contact with the contact interface and, based on the biometric output, measures the biological information.

2. The measurement apparatus according to claim 1, wherein the controller determines the contact condition based on a ratio of red components in the color information.

3. The measurement apparatus according to claim 2, wherein the controller determines the contact condition based on a change in the ratio of the red components.

4. (canceled)

5. The measurement apparatus according to claim 2, wherein, when the ratio of the red components is lower than a predetermined lower limit threshold and/or higher than a predetermined upper limit threshold, the controller causes the notification interface to issue a notification.

6. The measurement apparatus according to claim 1, wherein the controller controls the acquisition of the biometric output by the biological sensor based on the color information.

7. A measurement method for measuring biological information when a test site contacts with a contact interface, the measurement method comprising:
acquiring with a color information acquisition unit, color information of the test site in contact with the contact interface;
acquiring with a biological sensor, a biometric output from the test site; and
causing with a controller, based on the color information, a notification interface to notify of information about a contact condition of the test site in contact with the contact interface and, measuring, based on the biometric output, the biological information.
